(19) United States
(12) Patent Application Publication
(10) Pub. No.: US 2023/0047930 A1
(43) Pub. Date: Feb. 16, 2023

(54) METHODS AND ARRAYS FOR IDENTIFYING THE CELL OR TISSUE ORIGIN OF DNA

(71) Applicant: Ramot at Tel-Aviv University Ltd., Tel-Aviv (IL)

(72) Inventors: Yuval EBENSTEIN, Tel-Aviv (IL); Shahar ZIRKIN, Tel-Aviv (IL); Noa GILAT, Tel-Aviv (IL); Yael MICHAELI HOCH, Tel-Aviv (IL)

(73) Assignee: Ramot at Tel-Aviv University Ltd., Tel-Aviv (IL)

(21) Appl. No.: 17/788,003

(22) PCT Filed: Dec. 22, 2020
(86) PCT No.: PCT/IL2020/051320

§ 371 Date: Jun. 22, 2022

Related U.S. Application Data
(60) Provisional application No. 62/952,357, filed on Dec. 22, 2019.

(51) Int. Cl. C12Q 1/6881; C12Q 1/6886

(52) U.S. Cl. CPC C12Q 1/6881; C12Q 1/6886; C12Q 2600/154; C12Q 1/6837

(57) ABSTRACT

Methods and arrays for identifying the cell or tissue origin of DNA are provided. Accordingly there is provided a method of identifying DNA having a methylation pattern distinctive of a cell or tissue type or state comprising: labeling an epigenetic modification of interest in a DNA sample with a label; contacting said sample on an array comprising a plurality of probes for said DNA under conditions which allow specific hybridization between said plurality of probes and said DNA; and detecting said hybridization, wherein an amount of said label is indicative of the cell or tissue type or state, wherein the method is effected in the absence of amplification of said DNA.

### RELATED APPLICATION/S

This application claims the benefit of priority of U.S. Provisional Patent Application No. 62/952,357 filed on 22 Dec. 2019, the contents of which are incorporated herein by reference in their entirety.

### SEQUENCE LISTING STATEMENT

The ASCII file, entitled 84483 Sequence Listing.txt, created on 22 Dec. 2020, comprising 38,042,459 bytes, submitted concurrently with the filing of this application is incorporated herein by reference.

### FIELD AND BACKGROUND OF THE INVENTION

The present invention, in some embodiments thereof, relates to methods and arrays for identifying the cell or tissue origin of DNA.

Small fragments of cell-free circulating DNA (cfDNA) derived from apoptotic and necrotic cells (on average 5000 genome equivalents per ml) may be found in plasma. While the mechanisms underlying the release and clearance of cfDNA remain obscure, the phenomenon is rapidly being exploited for a variety of clinically relevant applications. Blood levels of cfDNA are known to increase under a variety of pathological conditions including cancer, autoimmune diseases, stroke and various organ injuries [e.g. 3-4]. Tumors are known to release DNA (including tumor-specific somatic mutations) into the circulation, providing non-invasive means for diagnosing cancer, monitoring tumor dynamics and analyzing genomic evolution.

Despite having an identical nucleotide sequence, the DNA of each cell type in the body carries unique epigenetic signature correlating with its gene expression profile. In particular, DNA methylation, serving to repress gene expression, is a fundamental aspect of tissue identity. Methylation patterns are unique to each cell type, conserved among cells of the same type in the same individual and between individuals, and are highly stable under physiologic or pathologic conditions. Therefore, it may be possible to use the DNA methylation pattern of cfDNA to determine its tissue of origin and hence to infer cell death in the source organ. The potential uses of a highly sensitive, minimally invasive assay of tissue specific cell death include early, precise diagnosis as well as monitoring response to therapy in both a clinical and drug-development setting.

Up to date several methods have been developed for the quantification of epigenetic modifications based on cfDNA (see e.g. 8, 10, 11; US Patent Application Publication No. 2017/0121767; and International Patent Application Publication Nos. WO2006128192 and WO2011038507). However, these methods are either laborious or expensive to perform, or are inaccurate and insensitive enough to meet the requirements for clinical use. Specifically, bisulfite conversion followed by PCR amplification, although the most common method used for methylation profiling, suffers from many flaws, primarily severe degradation of the treated DNA, the need for large amounts of starting material and in many cases, biased representation of the amplified DNA fragments. Other methods that involve array slide for methylation mapping are based on bisulfite conversion and thus show great bias and are also extremely expensive for clinical use.

Additional background art includes:

International Patent Application Publication Nos: WO2018/029693, WO2017/081689 and WO2014/191981;

Susan M Mitchell et al. BMC Cancer. 2014; 14: 54;

Daniel E. Deatherage et al. Methods Mol Biol. 2009; 556: 117-139;

Chun-Xiao Song et al. Cell Research 2017; 27: 1231-1242;

Wenyuan Li et al. Nucleic Acids Research, 2018; 46(15): e89;

Axel Schumacher et al. Nucleic Acids Res. 2006; 34(2): 528-542;

Javier Soto et al. Translational Research, 2016; 169: 1-18; and

Zahra Taleat et al. Trends in Analytical Chemistry, 2015; 66: 80-89.

### SUMMARY OF THE INVENTION

According to an aspect of some embodiments of the present invention there is provided a method of identifying DNA having a methylation pattern distinctive of a cell or tissue type or state, the method comprising:

(a) labeling an epigenetic modification of interest in a DNA sample with a label;

(b) contacting the sample on an array comprising a plurality of probes for the DNA under conditions which allow specific hybridization between the plurality of probes and the DNA; and

(c) detecting the hybridization, wherein an amount of the label is indicative of the cell or tissue type or state, wherein the method is effected in the absence of amplification of the DNA.

According to some embodiments of the invention, the epigenetic modification of interest is represented by a plurality of different DNA fragments.

According to some embodiments of the invention, the array is designed such that a plurality of different probes for the DNA are positioned on a single grid cell of the array.

According to some embodiments of the invention, the plurality of different probes comprise a plurality of different probes for the plurality of different DNA fragments.

According to an aspect of some embodiments of the present invention there is provided a method of identifying DNA having a methylation pattern distinctive of a cell or tissue type or state, the method comprising:

(a) labeling an epigenetic modification of interest in a sample comprising DNA with a label such that the epigenetic modification of interest is represented by a plurality of different DNA fragments;

(b) contacting the sample on an array comprising probes for the DNA fragments under conditions which allow specific hybridization between the probes and the DNA, wherein the array is designed such that a plurality of different probes for the plurality of different DNA fragments are positioned on a single grid cell of the array; and

(c) detecting the hybridization, wherein an amount of the label per the single grid cell of the array is indicative of the cell or tissue type or state.

According to some embodiments of the invention, the method is effected in the absence of amplification of the DNA and the DNA fragments.

According to some embodiments of the invention, the method comprising fragmenting the DNA so as to obtain DNA fragments prior to the (b).

According to some embodiments of the invention, the DNA fragments are 1000-1500 nucleotides long.

According to some embodiments of the invention, the DNA fragments are 50-300 nucleotides long.

According to some embodiments of the invention, the DNA fragments are about 200 nucleotides long.

According to some embodiments of the invention, a concentration of the DNA in the sample is ≤10 ng/μl.

According to some embodiments of the invention, a concentration of the DNA in the sample is ≤0.005 ng/μl.

According to some embodiments of the invention, the labeling comprises fluorescently labeling.

According to some embodiments of the invention, the labeling comprises enzymatically labeling.

According to some embodiments of the invention the epigenetic modification of the DNA is DNA methylation and the label is a methylation-specific label.

According to some embodiments of the invention, the method is effected in the absence of bisulfite conversion and/or sequencing.

According to some embodiments of the invention, the DNA is cellular DNA.

According to some embodiments of the invention, the method comprising lysing the cells of the cellular DNA prior to the labeling.

According to some embodiments of the invention, the DNA is cell-free DNA (cfDNA).

According to some embodiments of the invention, the cell comprises a pathologic cell.

According to some embodiments of the invention, the pathologic cell is a cancerous cell, a cell associated with a neurological disease, a cell associated with an autoimmune disease or a grafted cell.

According to some embodiments of the invention, the pathologic cell is a cancerous cell.

According to some embodiments of the invention, the cell having been exposed to an agent selected from the group consisting of: chemotherapy, chemical treatment, radiation and DNA damaging agent.

According to an aspect of some embodiments of the present invention there is provided a method of diagnosing a pathology in a subject, the method comprising obtaining a biological sample of the subject and identifying DNA having a methylation pattern distinctive of a cell or tissue type or state according to the method, wherein presence and/or level above a predetermined threshold of the DNA having the methylation pattern distinctive of the cell or tissue type or state is indicative of a pathology associated with the cell or tissue in the subject.

According to an aspect of some embodiments of the present invention there is provided a method of treating a pathology in a subject in need thereof, the method comprising:

(i) diagnosing the pathology in the subject according to the method; and wherein the pathology is indicated

(ii) treating the pathology in the subject.

According to an aspect of some embodiments of the present invention there is provided a method of monitoring a treatment for a pathology in a subject in need thereof, the method comprising obtaining a biological sample of the subject and identifying DNA having a methylation pattern distinctive of a cell or tissue associated with the pathology according to the method, wherein a decrease above a predetermined threshold of the DNA having the methylation pattern distinctive of the cell or tissue following treatment as compared to same prior to treatment indicates efficacy of treatment of the pathology in the subject.

According to some embodiments of the invention, the sample is a body fluid sample.

According to an aspect of some embodiments of the present invention there is provided a method of detecting death of a cell or tissue of interest in a subject comprising determining whether cell-free DNA (cfDNA) comprised in a fluid sample of the subject is derived from the cell or tissue of interest, wherein the determining is effected by the method, wherein presence and/or level above a predetermined threshold of the DNA having a methylation pattern distinctive of the cell or tissue of interest is indicative of death of the cell or tissue of interest.

According to some embodiments of the invention, when death of the cell or tissue is associated with a pathology, the method further comprises diagnosing the pathology.

According to some embodiments of the invention, the pathology is cancer, neurological disease, autoimmune disease or graft injury.

According to some embodiments of the invention, the pathology is cancer.

According to some embodiments of the invention, the fluid is selected from the group consisting of blood, plasma, serum, sperm, milk, urine, saliva and cerebral spinal fluid.

According to some embodiments of the invention, the fluid is selected from the group consisting of blood, plasma and serum.

According to some embodiments of the invention, the cell type is selected from the group consisting of a hepatocyte, a cardiomyocyte, a pancreatic beta cell, a pancreatic exocrine cell, a neuronal cell, a pneumocyte, a podocyte, an endothelial cell, a lymphocyte, an adipocyte, an oligodendrocyte, a skeletal muscle cell and an intestinal epithelial cell.

According to some embodiments of the invention, the tissue is selected from the group consisting of liver tissue, colon tissue, heart tissue, pancreatic tissue, brain tissue, lung tissue, renal tissue, breast tissue, bladder tissue, prostate tissue, blood tissue, thyroid tissue, ovarian tissue and spleen tissue.

According to an aspect of some embodiments of the present invention there is provided an array comprising a plurality of probes for a plurality of different nucleic acid sequences positioned on a single grid cell of the array.

According to some embodiments of the invention, the nucleic acid comprises DNA.

According to some embodiments of the invention, the plurality of different nucleic acid sequences comprise an epigenetic modification.

According to some embodiments of the invention, the plurality of different probes positioned on a single grid cell of the array comprise 2-100 different probes.

According to some embodiments of the invention, the plurality of different probes positioned on a single grid cell of the array comprise 5-50 different probes.

According to some embodiments of the invention, the concentration of the DNA in the sample is <10 ng/μl.

According to some embodiments of the invention, the concentration of the DNA in the sample is <0.005 ng/μl.

According to some embodiments of the invention, the concentration of the DNA in the sample is <10 fg/μl.

According to some embodiments of the invention, the concentration of the DNA in the sample is >1 fg/μl.

According to some embodiments of the invention, the array comprises a glass having a thickness <250 μm.

According to some embodiments of the invention, the array comprises a glass featuring a functionalized group capable of binding the plurality of probes.

According to some embodiments of the invention, the glass is coated with a silane layer comprising the functionalized group.

According to some embodiments of the invention, a thickness of the layers no more than 200 nm.

According to some embodiments of the invention, the glass is coated with a silane layer featuring the plurality of functional groups.

According to some embodiments of the invention the functional group(s) is/are capable of covalently binding said probe.

According to some embodiments of the invention the functional group(s) is/are an epoxide.

According to some embodiments of the invention, the array allows the use of an oil immersion microscope objective for imaging of the array.

According to some embodiments of the invention, the labeling comprises fluorescently labeling.

According to some embodiments of the invention, the labeling comprises enzymatically labeling.

According to some embodiments of the invention, the epigenetic modification of the DNA is DNA methylation and the label is a methylation-specific label.

According to some embodiments of the invention, the method is effected in the absence of bisulfite conversion and/or sequencing.

According to some embodiments of the invention, the DNA is cellular DNA.

According to some embodiments of the invention, the method comprises lysing the cells of the cellular DNA prior to contacting.

According to some embodiments of the invention, the DNA is cell-free DNA (cfDNA).

According to some embodiments of the invention, the cell comprises a pathologic cell.

According to some embodiments of the invention, the pathologic cell is a cancerous cell, a cell associated with a neurological disease, a cell associated with an autoimmune disease or a grafted cell.

According to some embodiments of the invention the pathologic cell is a cancerous cell.

According to some embodiments of the invention the cell having been exposed to an agent selected from the group consisting of: chemotherapy, chemical treatment, radiation and DNA damaging agent.

According to an aspect of some embodiments of the present invention there is provided a kit comprising the array; and a label, a positive control template comprising the nucleic acid sequences and/or an enzyme for labeling the nucleic acid sequences.

According to some embodiments of the invention, the positive control template comprises DNA having a methylation pattern distinctive of a cell or tissue type or state.

According to some embodiments of the invention, the label is fluorescent.

According to some embodiments of the invention, the label is for an epigenetic modification.

According to some embodiments of the invention, the array or the kit is for identifying a source of DNA in a sample.

According to some embodiments of the invention, the array or the kit is for diagnosing a pathology or monitoring a treatment of a pathology.

According to some embodiments of the invention, the epigenetic modification comprises unmethylated CpG.

According to some embodiments of the invention, the epigenetic modification comprises 5-methylcytosine (5mC) and/or 5-hydroxymethylcytosine (5hmC).

According to an aspect of some embodiments of the present invention there is provided a method of identifying presence and/or level of nucleic acid sequences in a sample comprising nucleic acids, the method comprising contacting the sample with the array under conditions which allow specific hybridization between the probes and the nucleic acid sequences.

According to some embodiments of the invention, the method comprising labeling the nucleic acid sequences with a label prior to the contacting.

According to some embodiments of the invention, the method comprising detecting the hybridization, wherein an amount of the label is indicative of the presence and/or level of the nucleic acid sequences in the sample.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation demonstrating methylation pattern of conceptual GeneX in cell free DNA (cfDNA). GeneX is unmethylated in liver but methylated in all other tissues (black tags mark methylated DNA. In a healthy state, all organs contribute a low baseline amount of cfDNA from GeneX. In a liver cancer state, the liver tumor releases large amounts of the unmethylated GeneX cfDNA which becomes dominant.

FIGS. 2A-C show schematic representation of DNA micro-arrays that can be used with some embodiments of the invention. FIG. 2A shows exemplary schematic representation of a DNA microarray used to diagnose cancer based on DNA methylation pattern. FIGS. 2B-C show schematic representations of probes localization in a microarray. FIG. 2B demonstrates a traditional microarray, wherein each of the spots on the array contains a single type of probe, allowing for the hybridization of only one type of DNA fragment (red stars represent fluorescent labeling of DNA). FIG. 2C demonstrates a microarray designed by the present inventors, wherein each spot is composed out of 2-100 different DNA probes, designed to capture different fragments of DNA originating from the same organ. This design allows for the enhancement of very low signals, such as in the case of cfDNA.

FIG. 3 is a schematic representation of the method developed for analyzing cfDNA from an individual with liver cancer as compared to a healthy individual. Briefly, cfDNA is recovered from blood drawn in a routine procedure. Upon extraction, all cfDNA is labeled with a fluorescent tag for unmethylated DNA using chemo-enzymatic reactions. In healthy individuals (upper panel), there is no specific organ or tissue with increased amounts of cfDNA, therefore, the mostly methylated cfDNA is not labeled and no visible signal appears. In the case of a liver cancer patient (lower panel), circulating tumor DNA originating from the liver is dominant in the total cfDNA. Hence, upon labeling and loading the cfDNA onto the microarray, the liver-specific spot fluoresces, indicating an abnormality in the patient's liver. FIGS. 4A-C demonstrate the feasibility and sensitivity of the hybridization procedure.

FIG. 4A is a fluorescent microscope image demonstrating the chemoenzymatic labeling procedure of cfDNA. Green dots represent cfDNA dyed with YOYO-1. Red dots represent the epigenetic labeling of unmethylated CpG sites (ATTO-647). Yellow dots represent colocalized red-green dots and demonstrate a cfDNA fragment containing one or more unmethylated CpG sites. FIGS. 4B-C show pictures of a custom designed microarray. In FIG. 4B, 5 ng of PCR amplified AGAP1 gene were fluorescently labeled and captured onto a specific AGAP1 complementary probe, on a custom designed microarray. The blue rectangles on the magnified area represent different regions on the array, which contain different probes. The AGAP1 labeled DNA hybridized only to its complementary probes. In FIG. 4C, 20 ng of cfDNA (extracted from 1 ml plasma) were labeled and loaded on to a custom microarray. The 4 spots encapsulated in the red rectangle represent the successful hybridization of the cfDNA to a specific DNA sequence (in this case, to the gene promotor of REG1A). The blue rectangles represent different spots, not visible since they did not capture labeled cfDNA.

FIGS. 5A-B demonstrate the feasibility of the hybridization procedure for diagnosing cancer. FIG. 5A shows fluorescence microscopy images from an array hybridized with DNA from a healthy individual as compared to an array hybridized with DNA from a colon cancer patient. The fluorescent 5hmC labels are shown in red, the array spots in gray. FIG. 5B is a graph demonstrating average intensities of the 67 spots that showed the strongest intensity difference between healthy individuals and cancer patients.

FIGS. 6A-D show representative images of single microscope field of views (FOVs) of the designed coverslips comprising PTP capture probes at a concentration of 300 ng/μl following hybridization with fluorescently labeled complementary (PTP-Alexa647N, FIGS. 6A-B) or non-complementary (NXEP4-Alexa647N, FIGS. 6C-D) DNA target samples, at a concentration of 10 fg/μl (FIGS. 6A and 6C) or 1 fg/μl (FIGS. 6B and 6D). Each lighted spot represents a single hybridized molecule. Scale bar=4 μm.

FIG. 7 is a graph summing several FOVs acquired for each of the indicated samples and analyzed by a custom software that counts the number of spots in each image using a specified threshold.

### DESCRIPTION OF SPECIFIC EMBODIMENTS OF THE INVENTION

The present invention, in some embodiments thereof, relates to methods and arrays for identifying the cell or tissue origin of DNA.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The requirement for amplification of the DNA in a sample intended for analysis of epigenetic modifications constitutes an obstacle to the widespread application of this technology for identifying tissue origin and pathology, as well as monitoring progression of disease or response to therapy.

In an effort to overcome the inaccuracy and lack of sensitivity of current methods for quantification of DNA methylation, such as bisulfite conversion followed by PCR amplification, the present inventors have surprisingly uncovered highly sensitive methods for identifying and quantifying distinctive patterns of DNA methylation and other epigenetic modifications. Using the methods of the invention, the present inventors have been able to successfully distinguish between methylated and non-methylated sequences in single DNA molecules of a sample without the need for amplification of the DNA in the sample prior to analysis.

Thus, according to one aspect of the invention there is provided a method of identifying DNA having a pattern of epigenetic modification distinctive of a cell or tissue type or state, the method comprising:

(a) labeling an epigenetic modification of interest in a DNA sample with a label;

(b) contacting the sample on an array comprising a plurality of probes for the DNA under conditions which allow specific hybridization between the plurality of probes and the DNA; and

(c) detecting the hybridization, wherein an amount of the label is indicative of the cell or tissue type or state, wherein the method is effected in the absence of amplification of the DNA.

Herein, the phrase “epigenetic modification” or “epigenetic DNA modification” refers to modifications of DNA which do not affect the DNA sequence, that is, they do not comprise replacement of one standard nucleotide (A, C, G or T) with another such nucleotide.

Examples of epigenetic DNA modifications that may be detected according to embodiments of the invention include, without limitation, unmethylated CpG, a 5-methylcytosine residue and/or a 5-hydroxymethylcytosine residue (which may be regarded as epigenetic modifications of cytosine (C)), and DNA damage (e.g., DNA lesions), optionally single strand DNA damage (e.g., abasic sites (missing purine or pyrimidine base), single strand breaks, pyrimidine dimers such as cyclobutane dimers and/or 6-4 pyrimidone photoproducts, and oxidized nucleotides).

According to specific embodiments, the epigenetic modification is methylation, or de-methylation of DNA. In some embodiments, epigenetic modification of the DNA is detected by identifying methylated cytosine residues. Where the modification comprises demethylation (of otherwise methylated DNA), epigenetic modification is detected by identifying unmethylated cytosines at methylation sites, for example, unmethlylated CpG sites.

According to specific embodiments, the epigenetic modification is selected from the group consisting of unmethylated CpG, 5-methylcytosine (5mC) and 5-hydroxymethylcytosine (5hmC).

Epigenetic modification patterns, and especially methylation patterns, are unique to each cell type or tissue and can change during pathologic processes (e.g. cancer) and thus can be used to identify cell or tissue type or state. Details of pathological conditions associated with epigenetic modifications, and particularly with methylation and de-methylation of DNA are provided herein.

As used herein, the term “distinctive of a cell or tissue type” refers to the differentiation between cells or of multiple cell types-forming a tissue. Examples of cells include, but are not limited to a hepatocyte, a cardiomyocyte, a pancreatic beta cell, a pancreatic exocrine cell, a neuronal cell, a pneumocyte, a podocyte, an endothelial cell, a lymphocyte, an adipocyte, an oligodendrocyte, a skeletal muscle cell and an intestinal epithelial cell.

Also envisaged for the methods disclosed herein are stem cells, progenitor cells, differentiated and undifferentiated cells, pluripotent cells. Cells suitable for analysis with the disclosed methods include, but are not limited to fetal cells, embryonic cells, newborn, child, adolescent, adult and geriatric cells.

The term “tissue” refers to part of an organism consisting of cells designed to perform a function or functions. Examples of tissues include, but are not limited to, liver tissue (comprising e.g. hepatocytes, sinusoidal endothelial cells, phagocytic Kupffer cells and hepatic stellate cells), colon tissue (comprising e.g. simple columnar epithelial cells, enterocytes, Goblet cells, enteroendocrine cells, Paneth cells, microfold cells, cup cells and tuft cells), heart tissue, pancreatic tissue (comprising e.g. exocrine cells, alpha cells, beta cells and delta cells), brain tissue (comprising e.g. neuronal cell and glial cells), lung tissue (comprising e.g. pneumocyts, squamous epithelial cells, goblet cells and club cells), renal tissue (comprising e.g. glomerulus parietal cells, podocytes, proximal tubule brush border cells, loop of Henle thin segment cells, thick ascending limb cells, kidney distal tubule cells collecting duct principal cells, collecting duct intercalated cells and interstitial kidney cells), breast tissue (comprising e.g. epithelial cells, myoepithelial cells and milk-secreting cuboidal cells), retina, skin tissue (comprising e.g. keratinocytes, melanocytes, Merkel cells, and Langerhans cells, mechanoreceptors, endothelial cells, adipocytes and fibroblasts), bone (comprising e.g. osteocytes, osteoblasts and osteoclasts), cartilage, connective tissue, blood tissue (comprising e.g. red blood cells, white blood cells and platelets), bladder tissue (comprising e.g. smooth muscle cells and urothelium cells), prostate tissue (comprising e.g. epithelial cells, smooth muscle cells and fibroblasts), thyroid tissue (comprising e.g. follicular cells and parafollicular cells), ovarian tissue, spleen tissue, muscle tissue, vascular tissue, gonadal tissue, hematopoietic tissue.

In specific embodiments, the cell type is selected from the group consisting of a hepatocyte, a cardiomyocyte, a pancreatic beta cell, a pancreatic exocrine cell, a neuronal cell, a pneumocyte, a podocyte, an endothelial cell, a lymphocyte, an adipocyte, an oligodendrocyte, a skeletal muscle cell and an intestinal epithelial cell.

In some embodiments, the tissue is selected from the group consisting of liver tissue, colon tissue, heart tissue, pancreatic tissue, brain tissue, lung tissue, renal tissue, breast tissue, bladder tissue, prostate tissue, blood tissue, thyroid tissue, ovarian tissue and spleen tissue.

In specific embodiments, fluid is selected from the group consisting of blood, plasma, serum, sperm, milk, urine, saliva and cerebral spinal fluid. In particular embodiments the fluid is selected from the group consisting of blood, plasma, urine, tears and serum. In particular, bodily fluids, and most often, are used for detection of cell-free DNA (cfDNA).

It will be appreciated that the methods disclosed herein are suitable for highly sensitive detection of modifications of the type characteristic to epigenetic modifications of nucleic acids in any properly prepared sample, and not exclusively in biological samples, or of biological material. Thus, in some embodiments, the sample is an aqueous sample of a nucleic acid which can be labeled and hybridized according to the methods disclosed herein.

As used herein, the term “distinctive of a cell or tissue state” refers to the differentiation between a healthy and a pathologic (e.g. cancerous) cell or tissue.

In some embodiments, pathological cells are cells from tissue affected by disease, including different cancers, autoimmune disorders, neurological disorders (Fragile X syndrome as well as Huntington, Alzheimer, and Parkinson diseases and schizophrenia).

Cancerous disease associated with epigenetic modifications, cells or tissue of which can be detected using the methods of the invention include, but are not limited to breast cancer, gastric cancer, liver cancer, esophageal cancer, acute myeloid leukemia, acute lymphocytic leukemia, chronic myeloid leukemia, chronic lymphoblastic leukemia, colorectal cancer and lung cancer. The following table shows some different types of cancer and the associated methylation modification target genes:

TABLE 1

| Cancer | | Promoter |
|---|---|---|
| type | Gene | methylation |
| Breast | RARB2, MSH2, ESR1B, AKR1B1, COL6A2, GPX7, HIST1H3C, | Hypermethylation |
| | HOXB4, RASGRF2, TM6SF1, ARHGEF7, TMEFF2, RASSF1, | |
| | BRCA1, STRATIFIN, RASSF1A | |
| Gastric | RUNX3 | Hypermethylation |
| Liver | CDKN2A | Hypermethylation |
| Esophageal | APC | Hypermethylation |
| Colorectal | SEPT9, hMLH1, CDKN2A/p16, HTLF, ALX4, TMEFF2/HPP1, | Hypermethylation |
| | NGFR, SFRP2, NEUROG1, RUNX3, UBE2Q1 | |
| Lung | RARB2, RASSF1A, CHFR, STRATI-FIN, SHOX2, RASSF1A APC1 | Hypermethylation |

Sequence specific as well as global modification of epigenetic profiles has been associated with autoimmune disease. Autoimmune diseases associated with epigenetic modifications, cells or tissue of which can be detected using the methods of the invention include, but are not limited to multiple sclerosis, systemic lupus erythematosus, asthma, Sjogren's syndrome, scleroderma, rheumatoid arthritis, primary biliary cirrhosis, Type I diabetes, psoriasis and ulcerative colitis.

Epigenetic modifications have been recognized in genes associated with development and disease of the nervous system, and in particular, the brain. Neurodegenerative and psychological disorders associated with epigenetic modifications, cells or tissue of which can be detected using the methods of the invention include, but are not limited to Alzheimer's disease, Huntington's disease, Fragile X syndrome, Autism and psychiatric diseases such as schizophrenia, Rubinstein-Taybi syndrome, bipolar, dementia, alcoholism and addiction, Tatton-Brown, overgrowth syndromes.

The term “label” or “labeling agent” refers to a detectable moiety which can be attached to DNA. Exemplary labels which are suitable for use with specific embodiments include, but are not limited to, a fluorescent agent, a radioactive agent, a magnetic agent, a chromophore, a bioluminescent agent, a chemiluminescent agent, a phosphorescent agent and a heavy metal cluster, as well as any other known detectable agents.

According to specific embodiments, the label is detectable by spectrophotometric measurements, and/or which can be utilized to produce optical imaging. Such labels include, for example, chromophores, fluorescent agents, phosphorescent agents, and heavy metal clusters.

As used herein, the term “chromophore” refers to a chemical moiety that, when attached to another molecule, renders the latter colored and thus visible when various spectrophotometric measurements are applied.

The phrase “fluorescent agent” refers to a compound that emits light at a specific wavelength during exposure to radiation from an external source.

The phrase “phosphorescent agent” refers to a compound emitting light without appreciable heat or external excitation as by slow oxidation of phosphorous.

A heavy metal cluster can be for example a cluster of gold atoms used, for example, for labeling in electron microscopy techniques (e.g., AFM).

The term “bioluminescent agent” describes a substance which emits light by a biochemical process.

The term “chemiluminescent agent” describes a substance which emits light as the result of a chemical reaction.

According to some embodiments of the invention, the label is a fluorescent labeling agent.

A fluorescent label can be a protein, quantum dots or small molecules. Common dye families include, but are not limited to Xanthene derivatives: fluorescein, rhodamine, Oregon green, eosin, Texas red etc.; Cyanine derivatives: cyanine, indocarbocyanine, oxacarbocyanine, thiacarbocyanine and merocyanine; Naphthalene derivatives (dansyl and prodan derivatives); Coumarin derivatives; oxadiazole derivatives: pyridyloxazole, nitrobenzoxadiazole and benzoxadiazole; Pyrene derivatives: cascade blue etc.; BODIPY (Invitrogen); Oxazine derivatives: Nile red, Nile blue, cresyl violet, oxazine 170 etc.; Acridine derivatives: proflavin, acridine orange, acridine yellow etc.; Arylmethine derivatives: auramine, crystal violet, malachite green; CF dye (Biotium); Alexa Fluor (Invitrogen); Atto and Tracy (Sigma Aldrich); FluoProbes (Interchim); Tetrapyrrole derivatives: porphin, phtalocyanine, bilirubin; cascade yellow; azure B; acridine orange; DAPI; Hoechst 33258; lucifer yellow; piroxicam; quinine and anthraqinone; squarylium; oligophenylenes; and the like.

Other fluorophores include: Hydroxycoumarin; Aminocoumarin; Methoxycoumarin; Cascade Blue; Pacific Blue; Pacific Orange; Lucifer yellow; NBD; R-Phycoerythrin (PE); PE-Cy5 conjugates; PE-Cy7 conjugates; Red 613; PerCP; TruRed; FluorX; Fluorescein; BODIPY-FL; TRITC; X-Rhodamine; Lissamine Rhodamine B; Texas Red; Aliaphycocyanin; APC-Cy7 conjugates.

Alexa Fluor dyes (Molecular Probes) include: Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, and Alexa Fluor 790.

Cy Dyes (GE Heathcare) include Cyt, Cy3, Cy3B, Cy3.5, Cy5, Cy5.5 and Cy7.

Nucleic acid probes include Hoechst 33342, DAPI, Hoechst 33258, SYTOX Blue,

ChromomycinA3, Mithramycin, YOYO-1, Ethidium Bromide, Acridine Orange, SYTOX Green, TOTO-1, TO-PRO-1, TO-PRO: Cyanine Monomer, Thiazole Orange, Propidium Iodide (PI), LDS 751, 7-AAD, SYTOX Orange, TOTO-3, TO-PRO-3, and DRAQ5.

Cell function probes include Indo-1, Fluo-3, DCFH, DHR, SNARF.

Fluorescent proteins include Y66H, Y66F, EBFP, EBFP2, Azurite, GFPuv, T-Sapphire, Cerulean, mCFP, ECFP, CyPet, Y66W, mKeima-Red, TagCFP, AmCyan1, mTFP1, S65A, Midoriishi Cyan, Wild Type GFP, S65C, TurboGFP, TagGFP, S65L, Emerald, S65T (Invitrogen), EGFP (Ciontech), Azami Green (MBL), ZsGreen1 (Clontech), TagYFP (Evrogen), EYFP (Clontech), Topaz, Venus, mCitrine, YPet, Turbo YFP, ZsYellow1 (Clontech), Kusabira Orange (MBL), mOrange, mKO, TurboRFP (Evrogen), tdTomato, TagRFP (Evrogen), DsRed (Clontech), DsRed2 (Clontech), mStrawberry, TurboFP602 (Evrogen), AsRed2 (Clontech), mRFP1, J-Red, mCherry, HcRed1 (Clontech), Katusha, Kate (Evrogen), TurboFP635 (Evrogen), mPlum, and mRaspberry.

Exemplary fluorescent labels include, but are not limited to, Alexa fluor dyes, Cy dyes, Atto dyes, TAMRA dyes and the like.

Labeling a nucleic acid e.g. DNA molecule with the label may optionally be effected using suitable reagents, such as are known in the art.

It is to be noted that, according to specific embodiments, the label is attached to the nucleic acid e.g. DNA molecule by means of click chemistry and that the reagents used for the reaction are derivatives of the labeling agent, which include a reactive group.

In specific embodiments, the label is attached to the DNA molecule using glycosyltransferase and a modified cofactor (e.g. glucose modified with an azide group) to functionalize the DNA, and a label is then covalently attached to the DNA via the functional group. In some embodiments, following functionalization, the label is attached using a click reaction, optionally a copper-free click reaction.

In other embodiments, the label is attached via non-covalent association.

In specific embodiments, determination of the epigenetic modifications is effected in the absence of bisulfite conversion of the sample DNA.

According to specific embodiments, the label comprises a plurality of labels, each of the plurality of labels being selective for a different type of e.g. epigenetic DNA modification. In such embodiments, the different labels are optionally characterized by different absorption, excitation and/or emission wavelengths. In specific embodiments, methylated and de-methylated cytosine residues are labelled with fluorescent labels of green and red emission spectra, respectively.

According to specific embodiments, the method further comprises cleaning the surface of the array (e.g., so as to remove nucleic acid e.g. DNA molecules not hybridized to the probes) subsequently to contacting on the array, and prior to determining an amount of the label. In some embodiments, cleaning the array is effected by rinsing with a liquid, e.g., an aqueous liquid.

According to some embodiments of the invention, there is provided an array comprising a plurality of different probes for a plurality of different nucleic acid sequences positioned on a grid cell of the array. As used herein the term “array” refers to a plurality of probes attached to a microscopic solid surface in an addressable manner.

According to specific embodiments the probes are specific for DNA fragments comprising epigenetic modifications which are distinctive of a cell or tissue type or state. For example, the DNA fragments detected by the probes comprise sequences which are differentially methylated with respect to a second non-identical cell or tissue, thereby allowing identifying the methylation signature of the cell or tissue of interest.

According to specific embodiments, the solid surface is in a form of a slide (e.g., a glass slide, a plastic slide, a silicon slide), for example, a slide such as used for microscopic observation. The slide is optionally configured to be readable by a commercial optical slide reader.

In specific embodiments, the solid surface is in the form of a thin glass slide or cover slip. In some embodiments, the solid surface is a glass slide or coverslip having a thickness of less than or equal to 250 μm, or less than or equal to 200 μm. In some embodiments, the solid surface is a glass slide or coverslip 80-130 μm thick, 130-170 μm thick, 160-190 μm thick or 190-250 μm thick.

In some embodiments, the solid surface is functionalized to allow binding of the plurality of probes, i.e. is chemically modified so as to feature a plurality of functional groups capable of binding the plurality of (e.g. DNA) probes.

The functional groups are such that can bind the plurality of probes via covalent, electrostatic and/or any other chemical interaction. In exemplary embodiments, the functional groups can bind the probe via covalent interactions.

Any functional group that can chemically interact with a functional group of a probe is contemplated, including negatively-charged functional groups that can bind to positively-charged groups of the probe (e.g., amines, guanines, guanidines), positively-charged groups that can bind to negatively charged groups of the probe (e.g., phosphates, carboxylates).

The chemical interaction can be via any chemical pathway that leads to a bond formation, whereby the bond can be a covalent bond, an ionic bond (including hydrogen bonds), hydrophobic interactions, aromatic interactions and Van-der-Waals interactions. Preferably, the chemical interaction is such that leads to a covalent bond formation, via, for example, SN1, SN2, esterification, addition-elimination, Shiff-base formation, UV-coupling, UV-cross-linking, Michael's addition, etc.

The functional groups can be selected according to the functional groups present in the probe of choice and the chemical interaction of choice.

In exemplary embodiments, the solid surface is a glass slide or cover slip coated with a layer that features functional groups capable of binding the plurality of (e.g. DNA) probes.

In some embodiments, the coated layer is 10-300 nm in thickness. In some embodiments, the coated layer is 20-250 nm thick, 25-225 nm thick, 30-200 nm thick, 40-175 nm thick, 50-200 nm thick, 75-250 nm thick, 100-200 nm thick, 75-150 nm thick, 60-130 nm thick. In specific embodiments, the coated layer is no more than 100, no more than 120, no more than 150, no more than 180, no more than 200, no more than 220, no more than 250 nm thick. In particular embodiments, the coated layer is less than or equal to 200 nm thick.

The plurality of functional groups can be the same or can include two or more types of functional groups.

In exemplary embodiments, the layer is or comprises a silane or siloxane that features the functional groups of choice.

Exemplary functional groups include, but are not limited to, epoxide (which can covalently bind to amine groups of a probe via a nucleophilic reaction), aldehyde (which can covalently bind to amine groups of a probe via Shiff-base formation), amine (which can bind electrostatically to negatively-charged groups of a probe), carboxylate or carboxylate ion (which can bind electrostatically to positively-charged groups of a probe), N-hydroxy succinimide

(NHS; which can covalently bind to amine groups of a probe via addition-elimination reaction to form an amide bond), thiol (which can bind to thiol groups of a probe to form a disulfide bond), maleimide (which can bind to thiol groups of a probe to form an ester bond), phenylenediisothiocyanate (PDITC; which can covalently bind to amine groups of a probe).

The functional groups can also be avidin/strepavidin or biotin, which can bind to biotin- or avidin/streptavidin-containing probe, respectively, to form an affinity pair.

Any other functional group that is capable of chemically interacting with a functional group of the probe is contemplated.

In exemplary embodiments, the solid surface is a glass slide or coverslip functionalized by silanization and/or epoxide modification. In specific embodiments, the solid surface is a glass slide or coverslip, coated with a silane layer that features a plurality of epoxide groups.

According to specific embodiments, the array is designed as a grid divided into separate cells (also known as, “grid cells”, “spots” or “features”) which can be typically-observed using magnification means, e.g., a microscope.

In specific embodiments, the grid cells are round, 1-5 mm in diameter. In some embodiments, the grid cells are round, 1 mm, 2 mm, 3 mm, 4 mm or 5 mm in diameter. In particular embodiments, the grid cells are round, 2 mm in diameter. In particular embodiments, the grid cells are round, 10-300 μm in diameter.

According to specific embodiments, the grid cells are separated from each other by a space or a spacer of about 50 — 1000 μm.

According to specific embodiments, the grid cells are separated from each other by a space or a spacer of at about 500 μm.

According to specific embodiments, the array is a traditional solid-phase array wherein each grid cell comprises identical probes.

According to other specific embodiments, the array is designed such that a plurality of different probes are positioned on a single grid cell.

As used herein “plurality of different probes positioned on a single grid cell” refers to non-identical probes directed at a plurality nucleic acid e.g. DNA target sequences mixed together in a single grid cell. In some embodiments, the plurality of different probes comprises a combination of DNA sequences having an epigenetic modification characteristic of a specific organ, tissue and/or state, distinguishing that organ, tissue and/or state from other organs, tissues and/or healthy states. In specific embodiments, genomic regions that are only unmethylated in specific organs and/or states are identified bioinformatically or by experimentation, and short sequences which are unmethylated (e.g. have reduced 5-hydroxymethyl cytosine- 5hmC) only in that specific organ and/or state, but are methylated (e.g. have normal amount of 5hmC) throughout other tissues and/or in a healthy state are provided as probes. In some embodiments, the plurality of different probes representing the specific organ, tissue and/or state is affixed to several grid cells. In other embodiments, the plurality of different probes representing the specific organ, tissue and/or state is affixed to a single grid cell. In other embodiments, a DNA microarray is designed wherein each grid cell represents a different organ or tissue and/or state.

In some embodiments, the plurality of different probes is designed to detect the epigenetic signature of a specific organ, tissue and/or state, or of a particular modification of a nucleic acid, by including multiple distinct sequences complementary to different fragments of DNA originating from the same organ, tissue and/or representing a specific state or particular modification of the nucleic acid on the same single grid cell. In some embodiments, the single grid cell comprises about 2-100 probes. In some embodiments, the single grid cell comprises probes representing 2-100 different sequences, 5-80 different sequences, 7-60 different sequences, 10-50 different sequences, or 12-40 different sequences originating from the same organ, tissue and/or representing a specific state. In some embodiments, the single grid cell comprises 2, 3, 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 110, about 120, about 130, about 140 or about 150 different sequences originating from the same organ, tissue and/or representing a specific state.

The sample is contacted with the array under conditions which allow specific hybridization between the probes and the nucleic acid e.g. DNA molecules. In specific embodiments, the sample comprises DNA labeled according to the methods described hereinabove.

In some embodiments, the sample comprises a tissue sample (e.g. biopsy) or a sample of a body fluid from a subject including but not limited to tissue biopsy, tissue section, formalin fixed paraffin embedded (FFPE) specimens, blood, plasma, serum, bone marrow, cerebro-spinal fluid, tears, sweat, lymph fluid, saliva, nasal swab or nasal aspirate, sputum, bronchoalveolar lavage, breast aspirate, pleural effusion, peritoneal fluid, glandular fluid, amniotic fluid, cervical swab or vaginal fluid, ejaculate, semen, prostate fluid, urine, pus, conjunctival fluid, duodenal juice, pancreatic juice, bile, and stool. In specific embodiments, the sample comprises DNA. In some embodiments, the sample comprises DNA extracted from a tissue or cells or body fluid. In particular embodiments, the sample comprises cell-free DNA (cfDNA). In specific embodiments, the sample is a serum or plasma sample comprising cfDNA, and the DNA of the sample is cfDNA.

DNA can also be isolated and purified by using commercially available DNA extraction kits such as QiaAmp tissue kits. Body fluid should be pre-treated under appropriate condition prior to DNA extraction. For example, if a blood sample is used in this invention, anti-coagulants contained in whole blood should be able to inhibit DNAse activity. A suitable anti-coagulant may be a chelating agent such as EDTA that prevents both DNAse-caused DNA degradation and clotting of the whole blood samples. If other body fluid samples such as sputum are used, Cells in these kinds of samples can be collected by the procedures described in prior art. For example, collection of cells in a urine sample can simply be achieved by simply centrifugation, while collection of cells in a sputum sample requires DTT treatment of sputum followed by filtering through a nylon gauze mesh filter and then centrifugation. If a stool sample is used, a stool stabilizing and homogenizing reagents should be added to stabilize DNA and remove stool particles. Human DNA fraction from total stool DNA then can be primarily isolated or purified using commercially available stool DNA isolation kits such as Qiagen DNA Stool Mini Kit (using the protocol for human DNA extraction) or be captured by methyl-binding domain (MBD)-based methylated DNA capture methods after total DNA isolation [Zhou H et al., Clinical Chemistry, 2007].

In some embodiments, the sample comprises cells and/or tissues, and DNA of the sample is cellular DNA (e.g. genomic DNA). Cellular DNA can be obtained after its release from the cell. In some embodiments, cells are disrupted mechanically (e.g. sonication, pressure, impact—e.g. glass beads, etc), chemically (detergents such as SDS, Triton, etc) or thermally (heating). In some embodiments, the cellular contents are then subject to denaturation of nucleoproteins and/or inactivation of cellular enzymes, for example, by guanidinium thiocyanate, phenol extraction, proteinase, chelation and/or detergent treatment. Following denaturation/inactivation, in some embodiments, the cell lysate is further cleansed of contaminants, for example, by salting out, organic extraction, PEG extraction, chelation and/or adsorption (e.g. diatomaceous earth).

Finally, DNA may be precipitated from the cell lysate for purification. Methods for precipitation of DNA include, but are not limited to alcohol (e.g. ethanol, isopropanol) precipitation, sodium acetate +alcohol, and magnetic beads (DNA can be adsorbed onto silica-coated surfaces). DNA can then be processed for detection of profiles of epigenetic modifications according to the methods of the invention.

It will be appreciated that, in some embodiments, the target DNA for analysis is cell-free DNA (cfDNA). In such cases, either tissue or cellular components are removed from the samples, leaving cfDNA, or the samples are processed for characterization of the profile of epigenetic modification without removal of cells or cellular debris, for example, when the sample is of a bodily fluid.

In some embodiments, the DNA of the sample is in DNA fragments. The DNA fragments can be in the range of 20-2000 nucleotides in length. In some embodiments, the DNA fragments of the sample are 50-1500 nucleotides long, 100-1200 nucleotides long, 150- 1000 nucleotides long, 1000-1500 nucleotides long, 50-300 nucleotides long, about 100, about 200, about 300, about 400, about 500, about 600, about 700, about 800, about 900, about 100, about 1100, about 1200, about 1300, about 1400, about 1500, about 1600, about 1700, about 1800, about 1900 or about 2000 nucleotides long. In specific embodiments, the DNA fragments are 1000-1500 nucleotides long, 50-300 nucleotides long or about 200 nucleotides long.

In some embodiments, the DNA of the sample is fragmented prior to contacting the sample in the array. Fragmenting the DNA of a sample can be effected by methods known in the art, including but not exclusively enzymatic (e.g. endonuclease) fragmentation, acoustic fragmentation, sonication, centrifugal shearing, point-sink shearing, needle (hypodermic) shearing and the like. In specific embodiments, the DNA is fragmented by shearing. Some methods of DNA fragmentation are detailed in PCT Publication WO 2016/178207.

In some embodiments, the epigenetic modification of interest (e.g. methylation of cytosine at CpG, 5hmc) in the DNA of the samples is present on a plurality of different fragments of the sample DNA. In some specific embodiments, the plurality of different DNA probes which can hybridize and thus detect the epigenetic modification of the sample DNA, are bound to the array or grid in a single cell of the grid or array. Thus, in some embodiments, there is provided a method of identifying DNA having a pattern of epigenetic modification distinctive of a cell or tissue type or state, the method comprising: labeling an epigenetic modification of interest in a sample comprising DNA with a label such that the epigenetic modification of interest is represented by a plurality of different DNA fragments; contacting the sample on an array comprising probes for the DNA fragments under conditions which allow specific hybridization between the probes and the DNA, wherein the array is designed such that a plurality of different probes for the plurality of different DNA fragments are positioned on a single grid cell of the array; and detecting the hybridization, wherein an amount of the label per each single grid cell of said array is indicative of the cell or tissue type or state.

The present inventors have surprisingly found that, using the methods of the invention, samples containing extremely small concentrations of DNA can be analyzed, without need for amplification of the DNA in the sample, in the ranges of a few nanograms per microliter (ng/μl) sample, and even within the range of femtograms per microliter (fg/μl).

Thus, in some embodiments, the concentration of DNA in the sample is in the range of 0.1-100 ng/μl, less than or equal to 50, 40, 30, 20, 10, 1, 0.9, 0.8, 0.7, 0.6, 0.5, 0.4, 0.3, 0.2 or 0.1 ng/μl. In some embodiments, the concentration of DNA in the sample is in the range of 1-10, 0.5-10, 0.1-10, 2-15, 2-20, 1-50, 1-25, 5-50, 2-35, 5-40, 20-80, 10-60 and 25-75 ng/μl.

In other embodiments, the concentration of DNA in the sample is in the range of 0.1-100 pg/μl, less than or equal to 50, 40, 30, 20, 10, 1, 0.9, 0.8, 0.7, 0.6, 0.5, 0.4, 0.3, 0.2 or 0.1 pg/μl. In some embodiments, the concentration of DNA in the sample is in the range of 1-10, 0.5-10, 0.1-10, 2-15, 2-20, 1-50, 1-25, 5-50, 2-35, 5-40, 20-80, 10-60 and 25-75 pg/μl.

In still other embodiments, the concentration of DNA in the sample is in the range of 0.1-100 fg/μl, less than or equal to 50, 40, 30, 20, 10, 1, 0.9, 0.8, 0.7, 0.6, 0.5, 0.4, 0.3, 0.2 or 0.1 fg/μl. In some embodiments, the concentration of DNA in the sample is in the range of 1-10, 0.5-10, 0.1-10, 2-15, 2-20, 1-50, 1-25, 5-50, 2-35, 5-40, 20-80, 10-60 and 25-75 pg/μl.

In specific embodiments, the concentration of DNA in the sample is equal to, or less than 10 ng/μl. In other specific embodiments, the concentration of DNA in the sample is equal to or less than 0.005 ng/μl. In yet other specific embodiments, the concentration of DNA in the sample is equal to or less than 10 fg/μl.

As used herein, “hybridization conditions” refer to conditions that promote specific annealing of the probe with its specific nucleic acid e.g. DNA target sequence. Such conditions are well-known in the art and include, but not limited to, temperature, buffer, salt, ionic strength, pH, time and the like. Various considerations must be taken into account when selecting the stringency of the hybridization conditions. For example, the more closely the probe reflects the target nucleic acid sequence, the higher the stringency of the assay conditions can be, although the stringency must not be too high so as to prevent hybridization of the probes to the target sequence. Further, the lower the homology of the probes to the target sequence, the lower the stringency of the assay conditions should be, although the stringency must not be too low to allow hybridization to non-specific nucleic acid sequences. The ability to optimize the reaction conditions is well within the knowledge of one of ordinary skill in the art.

Generally, annealing temperature and timing are determined both by the efficiency with which a probe is expected to anneal to the target and the degree of mismatch that is to be tolerated. The temperature generally ranges from about 37° C. to about 50° C., and usually from about 40° C. to about 45° C. Annealing conditions are generally maintained for a period of time ranging from about 1 minute to about 30 minutes, usually from about 1 minute to about 10 minutes.

According to specific embodiments, the hybridization conditions comprise a denaturation step in order to dissociate any double-stranded or hybridized nucleic acid present in the reaction mixture prior to the annealing. The denaturation step generally comprises heating the reaction mixture to an elevated temperature and maintaining the mixture at the elevated temperature for a sufficient period of time. For denaturation, the temperature of the reaction mixture is usually raised to, and maintained at, a temperature ranging from about 85° C. to about 100° C., usually from about 90° C. to about 98° C., and more usually from about 93° C. to about 96° C. for a period of time ranging from about 1 to about 30 minutes, usually from about 5 to about 10 minutes.

In specific embodiments, hybridization of a dsDNA sample comprises incubating the grid or microarray in a pre-hybridization buffer (20 × SSC, 20% SDS, 5% BSA) for 20 minutes at 65° C., placing the dsDNA sample in a hybridization solution (20× SSC, 20% SDS), incubating for 5 minutes pre-hybridization in 95° C. to denature the sample DNA, heating the grid or microarray in a thermos-shaker to 42° C., followed by immediate addition of the denatured (by incubation at 95° C.) dsDNA sample.

According to specific embodiments, the method is effected on a non-amplified nucleic acid e.g. DNA sample e.g., not subjected to any amplification prior to the labeling.

According to specific embodiments, the method is effected without amplification of the nucleic acid e.g. DNA following labeling.

According to specific embodiments, the method is effected on a nucleic acid e.g. DNA sample not subjected to any amplification prior to fragmentation.

According to specific embodiments, the method is effected without amplification of the nucleic acid e.g. DNA following fragmentation.

According to specific embodiments, the method is effected without amplification of the nucleic acid e.g. DNA prior to contacting the sample on the array.

According to specific embodiments, the method is effected in the absence of amplification; i.e. in the absence of any amplification of the nucleic acid e.g. DNA at any stage prior to the labeling up to the contacting with the array.

As used herein, the term “amplification” refers to a process that increases the representation of a population of specific nucleic acid sequences in a sample by producing multiple (i.e., at least 2) copies of the desired sequences. Methods for nucleic acid amplification are known in the art and include, but are not limited to, polymerase chain reaction (PCR) and ligase chain reaction (LCR). In a typical PCR amplification reaction, a nucleic acid sequence of interest is often amplified at least fifty thousand fold in amount over its amount in the starting sample. A typical amplification reaction is carried out by contacting a forward and reverse primer (a primer pair) to the sample DNA together with any additional amplification reaction reagents under conditions which allow amplification of the target sequence.

Following hybridization, the cells of the grid or array are washed to remove unhybridized DNA, and to allow detection of the pattern (patterns) of epigenetic modification (e.g. methylation and/or de-methylation) characterizing the cells/tissues/organs/fluids represented by the samples.

As detailed herein, in some embodiments the sample DNA is labeled for detection by fluorescent labelling. In other embodiments, the sample DNA is labelled by enzymatic labelling. In specific embodiments, the sample is labelled by enzymatic glucosylation of methylated cytosine residues followed by aldehyde formation via glucose oxidation and covalent linkage of the aldehyde moieties with the fluorescent label by oxime ligation.

Detection of labeled DNA following hybridization and washing of the cells (or spots) of the grid or array can be performed using any spectrophotometric, chemical and/or enzymatic methods. In specific embodiments, the label is a fluorescent label, and the labeled DNA is detected using a fluorescent microscope, in particular an epi-fluorescence microscope. In particular embodiments, detection of the fluorescent labels is performed using high power, oil-immersion microscope objectives (e.g. 100×) for imaging of the grid or array following hybridization and washing. Results of the detection can be processed and analyzed by any suitable statistical tools. In specific embodiments, several fields of view are obtained for analysis, and the number of fluorescent spots, and intensity, are analyzed by suitable computer image processing software and hardware.

Since the methods of the invention can be used to distinguish between cells, tissue, organs and/or states characteristic of certain pathologies, the methods for detection of DNA with patterns of epigenetic modifications can be used to diagnose pathology. It will be appreciated that the method disclosed herein can also be used for highly sensitive detection of modifications of nucleic acids characteristic of epigenetic changes on any sample of nucleic acids, or even on molecules other than nucleic acids. Thus, in an exemplary embodiments, the methods of detection disclosed herein can be used to detect methylation (or demethylation) events and/or methylation patterns in any sample comprising a methylated, or demethylated molecules capable of being labeled and binding to immobilized “capture” probes of an array or grid of cells.

Thus, in some embodiments there is provided a method of diagnosing a pathology in a subject, the method comprising identifying DNA in the sample having a pattern of epigenetic modification distinctive of a cell or tissue type or state according to the method of invention, wherein presence and/or level above a predetermined threshold of the DNA having the pattern of epigenetic modification distinctive of the cell or tissue type or state is indicative of a pathology associated with the cell or tissue in the subject.

The method can be carried out for diagnosing diseases which are associated with epigenetic modifications. In some embodiments, the method can be used to diagnose diseases or conditions associated with altered methylation status, including, but not limited to fascioscapulohumeral muscular dystrophy (FSHD) and cancers (see, for example, diseases associated with epigenetic modifications listed hereinabove). A non-limiting list of cancers which may be diagnosed using the methods described herein includes, but is not limited to Examples of cancer which can be diagnosed are summarized herein below.

Cancer

Non-limiting examples of cancers which can be diagnosed by the method of this aspect of some embodiments of the invention can be any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), oral squamous cell carcinoma (OSCC), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute - megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

As used herein, the term “diagnosing” refers to determining the presence or absence of a pathology (e.g. a disease, disorder, condition or syndrome), classifying a pathology or a symptom, determining a severity of the pathology, monitoring the pathology's progression, forecasting an outcome of the pathology and/or prospects of recovery and screening of a subject for a specific disease.

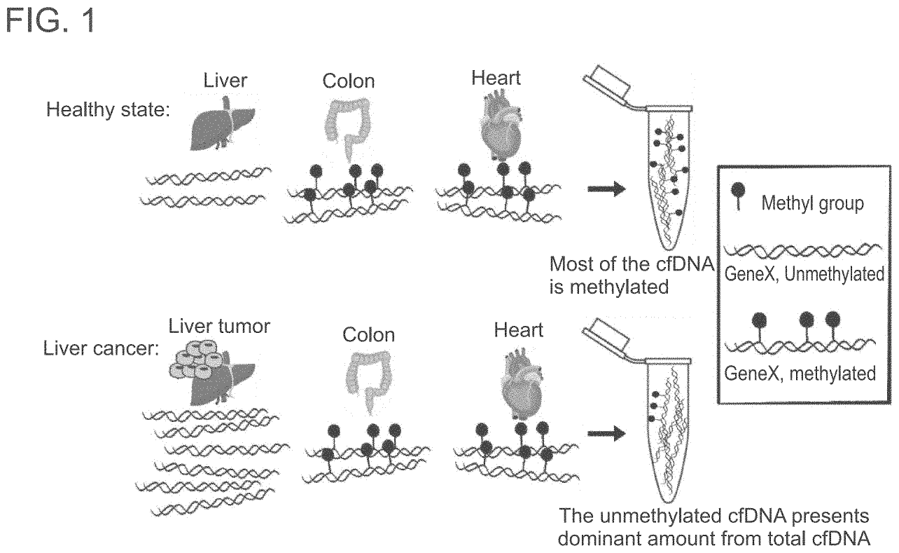

In some embodiments, the pattern of epigenetic modification distinctive of the cell and/or tissue associated with the pathology is a reduction in the extent of methylation of cancerous cells within a tumor (see FIGS. 1, 3 and 5), relative to that of healthy tissue. In some embodiments, the threshold for diagnosis of the pathological condition is expressed as a significant decrease in the amount of methylation of the DNA. As used herein “significant decrease” refers to a decrease that is statistically significant (e.g., P<0.05).

Typically, the decrease is subtle between the normal control and the pathogenic sample and therefore the sensitivity of the method of epigenetic modification detection is crucial.

According to a specific embodiment, the significant decrease is below 90%.

According to a specific embodiment, the significant decrease is below 75%.

According to a specific embodiment, the significant decrease is below 50%.

According to a specific embodiment, the significant decrease is between 5-45%.

According to a specific embodiment, the significant decrease is between 10-50%.

According to a specific embodiment, the significant decrease is between 10-45%.

According to a specific embodiment, the significant decrease is between 20-50%.

According to a specific embodiment, the significant decrease is between 20-45%.

According to a specific embodiment, the significant decrease is between 30-50%.

According to a specific embodiment, the significant decrease is between 30-45%.

According to a specific embodiment, the significant decrease is between 10-30%.

According to a specific embodiment, the significant decrease is between 1-30%.

According to a specific embodiment, the significant decrease is between 5-30%.

According to a specific embodiment, the significant decrease is between 1-50%.

According to a specific embodiment, the significant decrease is between 1-20%.

According to a specific embodiment, the significant decrease is between 1-10%.

In some cases, changes of greater degree are characteristic, with a difference of 1, 2, 5, 10 or more fold between the amount of epigenetic modification (e.g. methylation or demethylation) in the normal control and the pathogenic sample. Thus, in some embodiments, the significant decrease is in the range of 1- 100 fold.

According to a specific embodiment, the significant decrease is between 5-85 fold.

According to a specific embodiment, the significant decrease is between 7-60 fold.

According to a specific embodiment, the significant decrease is between 10-45 fold.

According to a specific embodiment, the significant decrease is between 20-50 fold.

According to a specific embodiment, the significant decrease is between 20-45 fold.

According to a specific embodiment, the significant decrease is between 30-50 fold.

According to a specific embodiment, the significant decrease is between 30-45 fold.

According to a specific embodiment, the significant decrease is between 10-30 fold.

According to a specific embodiment, the significant decrease is between 1-20 fold.

According to a specific embodiment, the significant decrease is between 5-30 fold.

According to a specific embodiment, the significant decrease is between 1-50 fold.

According to a specific embodiment, the significant decrease is between 1-10 fold.

According to a specific embodiment, the significant decrease is between 2-15 fold.

It will be appreciated that some pathologies, conditions and/or states are characterized by gain of epigenetic modification rather than reduction in the degree of epigenetic modification in the nucleic acid of the cells/tissue/organs (for example, hypermethylation of specific sites (e.g.

promoter sequences) in certain types of cancer- see Table 1 herein). Thus, in some embodiments, the pattern of epigenetic modification distinctive of the cell and/or tissue associated with the pathology is an increase in the extent of epigenetic modification (e.g. methylation) of cancerous cells within a tumor, relative to that of healthy tissue. In some embodiments, the threshold for diagnosis of the pathological condition is expressed as a significant increase in the amount of methylation of the DNA. As used herein “significant increase” refers to a decrease that is statistically significant (e.g., P<0.05).

According to a specific embodiment, the significant increase is above 95%.

According to a specific embodiment, the significant increase is between 75-95%.

According to a specific embodiment, the significant increase is between 50 and 75%.

According to a specific embodiment, the significant increase is between 5-45%.

In some cases, changes of greater degree are characteristic, with a difference of 1, 2, 5, 10 or more fold between the amount of epigenetic modification (e.g. methylation or demethylation) in the normal control and the pathogenic sample. Thus, in some embodiments, the significant increase is in the range of 1- 100 fold, 5-80 fold, 2-60 fold, 3-50 fold or greater.

In some embodiments, the methods described herein may be used for identifying a pre-malignant stage of cancer development in a cell, tissue or organ of the subject. As used herein “pre-malignant” refers to a tissue that is not yet malignant but is poised to become malignant. Appropriate clinical and laboratory studies are designed to detect premalignant tissue while it is still in a premalignant stage. Examples of premalignant growths include polyps in the colon, actinic keratosis of the skin, dysplasia of the cervix, metaplasia of the lung, pre-malignant lesions of oral squamous cell carcinoma (OSCC) and leukoplakia (white patches in the mouth).

Typically, the pre-malignant lesion has a prevalence of epigenetic modification which is intermediate between that of a healthy tissue and that of a cancerous tissue (e.g., all data is available from the same subject).

As used herein providing a DNA sample of a cell, tissue or organ of a subject, refers to a tissue biopsy.

The biopsy can be taken from a non-affected/suspected region (e.g., control), a region diagnosed with a disease (e.g. cancer) and/or a region suspected of being diseased or subject to a disease process (e.g. premalignant) and that can be in the vicinity of an affected region.

According to some embodiments of the invention, screening of the subject for a specific disease is followed by substantiation of the screen results using gold standard methods (e.g., biopsy, ultrasound, CT, MRI, TAA expression, cytomorphometry, clinical tissue staining (e.g., Vital iodine stain, Tblue stain)).

The methods described herein can also be used to treat a pathology in a subject. Employing the methods described herein for diagnosing a pathology (e.g. cancer) associated with alteration of epigenetic modification of cells and/or tissue in a subject, subjects diagnosed with such an epigenetic modification-associated pathology can be treated, according to the nature and severity of the pathology or condition.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The methods described herein may also be used for monitoring the state of a pathology in a subject, or monitoring a treatment for a pathology in a subject. Thus, there is provided a method of monitoring a treatment for a pathology in a subject in need thereof, the method comprising obtaining a biological sample of the subject and identifying DNA having a pattern of epigenetic modification distinctive of a cell or tissue associated with the pathology according to the methods described herein, wherein a decrease above a predetermined threshold of the DNA having the pattern of epigenetic modification distinctive of the cell or tissue following treatment as compared to the pattern of the DNA prior to treatment indicates efficacy of treatment of the pathology in the subject. Such monitoring can be performed at intervals following the treatment, and dosage and regimen adjusted according to the results of the monitoring.

Methylation patterns are highly stable under physiologic or pathologic conditions. Monitoring of tissue-specific DNA methylation markers in cfDNA has been shown effective for detection of cell death in specific tissues, including pancreatic β-cell death in type I diabetes, oligodendrocyte death in relapsing multiple sclerosis, brain cell death in patients after traumatic or ischemic brain damage, and exocrine pancreas cell death in pancreatic cancer or pancreatitis. Thus, the methods described herein may be used for determining death of a cell or tissue of interest, wherein the presence and/or level above a predetermined threshold of the DNA having a pattern of epigenetic modification distinctive of the cell or tissue of interest is indicative of death of the cell or tissue of interest.

cfDNA derives, for the most part, from dead cells, and blood levels of cfDNA are known to increase in many conditions, for example, traumatic brain injury, cardiovascular disease, sepsis and intensive exercise. Thus, in specific embodiments, the distinctive epigenetic patterns are discerned in the cfDNA of a sample or samples from the subject.

Also contemplated are a kit or kits comprising the grid or array described herein, a label, a positive control template comprising the nucleic acid sequences and/or an enzyme for labeling the nucleic acid sequences. In some embodiments, the positive control template comprises DNA having a pattern of epigenetic modification distinctive of a cell type or state. In some embodiments, the label of the kit is a fluorescent label, and is specific for the epigenetic modifications. Such kits may be used for identifying a source of DNA in a sample, diagnosis and/or treatment and/or monitoring of a pathology in a subject, or determination of organ or tissue-specific cell death.

The methods described herein can also be used to detect and quantify specific nucleic acid sequences in any sample comprising nucleic acids, not only biological samples for analysis of epigenetic modification of cellular or tissue DNA. Contacting any sample comprising nucleic acids which can hybridize with any of the sequences affixed (bound) to the array or grid, under conditions allowing hybridization of complementary sequences, and washing of unhybridized sequences can provide a basis for detection of such complementary nucleic acid sequences in any sample. In specific embodiments, the nucleic acid of the sample is labeled at specific sites prior to contact with the kit or array or grid. Detecting and quantifying the hybridization is effected as for the biological samples, as described herein.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “treating” and “treatment” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Experimental Procedures

The novel method developed by the present inventors takes advantage of the unique methylation pattern of DNA to determine tissue state, and more specifically relates to the methylation pattern of cell free DNA (cfDNA) originating from different cell-types, to determine the tissue of origin for the cfDNA fragments and/or to diagnose disease e.g. cancer [FIG. 1]. For example, during the progression of a tumor, cancer cells rapidly proliferate and expand over the infected tissue. This process is accompanied by the increase of apoptosis and necrosis of tumor cells, resulting in increased amounts of circulating, cell-free, tumor DNA. This DNA still carries some of the epigenetic signatures of the tissue it originated from, thereby allows associating it with its source organ [Figure 1]. On the other hand, cancer cells also differ in their epigenetic profile as compared to their healthy counterparts. For example, it has been shown that the modification 5-hydroxymethyl cytosine (5hmC) is downregulated in cancer cells. Even at early stages of carcinogenesis, circulating tumor DNA levels are rising, thus contributing a considerable amount of fragments to the total amount of cfDNA [12].

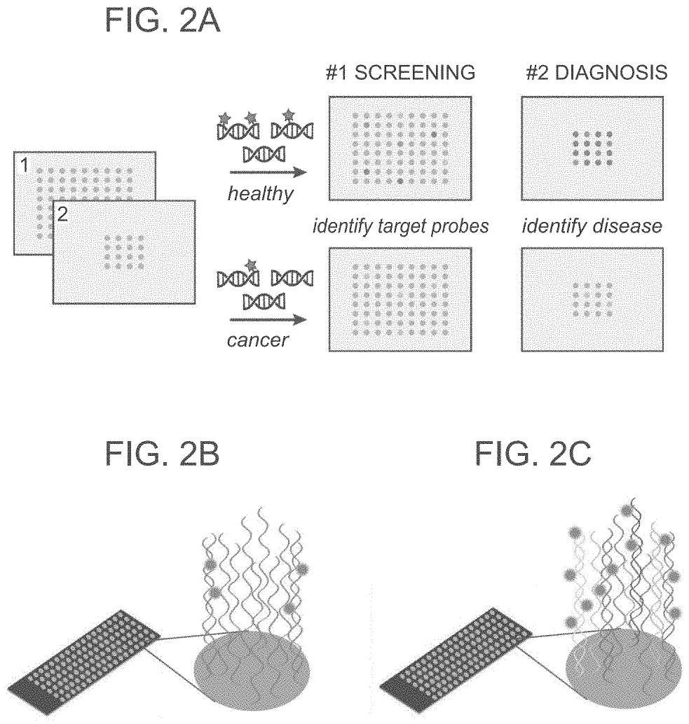

To this end genomic regions that are only unmethylated in specific organs and/or states are bioinformatically and/or experimentally mapped. For each organ and/or state a list of short sequences which are unmethylated only in that specific organ and/or state, but are methylated throughout all other tissues and/or in a healthy state is generated. Following, a commercial or custom-designed DNA microarray is used, wherein a combination of several spots represents a different organ or tissue and/or state (FIG. 2A). Alternatively, a DNA microarray is designed, wherein each spot represents a different organ or tissue and/or state.

Optionally, to overcome the low signal achieved from unamplified DNA, multiple distinct capture sequences are present on the same spot of the array (about 2-100 probes), designed to capture different fragments of DNA originating from the same organ. This allows different DNA regions to be captured on the same spot of the array, all of which are designed to detect the epigenetic signature of a specific organ [FIGS. 2B-C].

Following, DNA is extracted from the tissue of interest (e.g. plasma) using commercially available kits and fragmented into approximately 200-1300 bp pieces. Alternatively, cfDNA is extracted from blood or plasma using commercially available kits. Typically, cfDNA is fragmented and thus there is no need in a fragmentation step. However, optionally, the cfDNA is fragmented into approximately 200 bp pieces. Fluorescent labeling of unmethylated CpGs is performed by a newly developed chemoenzymatic reaction [13-14 and Michaeli et al. Chem Commun (Camb). (2013) 49(77):8599-601]. A CpG methyltransferase is used in-vitro together with a synthetic cofactor to attach a fluorophore to the unmethylated site. As explained, specific target sequences in the genome are unmethylated in each organ/state; hence only this unmethylated DNA e.g. cfDNA, originating from the specific organ/state, are labeled; as opposed to DNA e.g. cfDNA originating from all other organs/states which is methylated at these loci. Next, the labeled double-stranded DNA (dsDNA) is hybridized to a commercial or custom designed DNA microarray, which contains the capture probes of interest, as explained above.

Most known assays for microarray hybridization use either RNA or ssDNA. The present inventors have developed a temperature cycling protocol for high-yield hybridization of dsDNA; and thus avoid additional steps to turn dsDNA into ssDNA. The designed probes contain ssDNA copies of a DNA strand, complementary to one strand of the DNA e.g. cfDNA fragment of interest. In the hybridization process, DNA e.g. cfDNA is heated and denaturated into single-strands, followed by cooling and subsequent hybridization either back to its complementary strand, or to the designed probes. In order to favor hybridizing of DNA e.g. cfDNA to the probes, slide surface chemistry and physical properties is examined, the spot size is adjusted, and probe concentration and blocking buffer are optimized. The developed protocol used with specific embodiments of the invention for dsDNA hybridization includes the following steps:
1. Incubating the microarray in a pre-hybridization buffer (20 × SSC, 20% SDS, 5% BSA) for 20 minutes in 65° C.
2. Placing the dsDNA in a hybridization solution (20 × SSC, 20% SDS), incubating for 5 minutes pre-hybridization in 95° C.
3. Heating the microarray in a thermos-shaker to 42° C., followed by immediate addition of the dsDNA (following incubation in 95° C.).

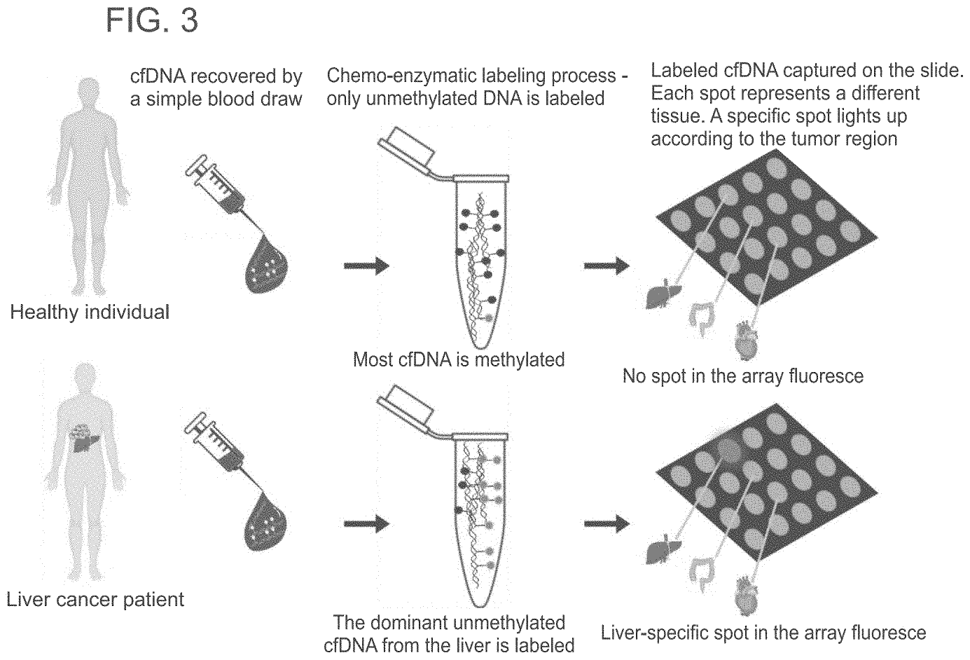

Finally, the microarray is imaged using a commercial slide scanner or a microscope. The fluorescent pattern is indicative of the cell or tissue type and/or state. For example, when hybridizing a labeled cfDNA to a microarray in which each spot represents a single organ, a fluorescent signal in a specific spot on the microarray, indicates that the organ represented by this spot released higher quantities of cfDNA. Hence, for example, when analyzing cfDNA from a healthy individual, the microarray does not display any abnormal signal since no organ is releasing abnormal amounts cfDNA [Figure 3 upper panel]. However, when applying the same assay on cfDNA from a liver cancer patient, the increased amounts of circulating tumor DNA originating from the liver in this individual, lights-up the array spot representing the liver [FIG. 3 lower panel].

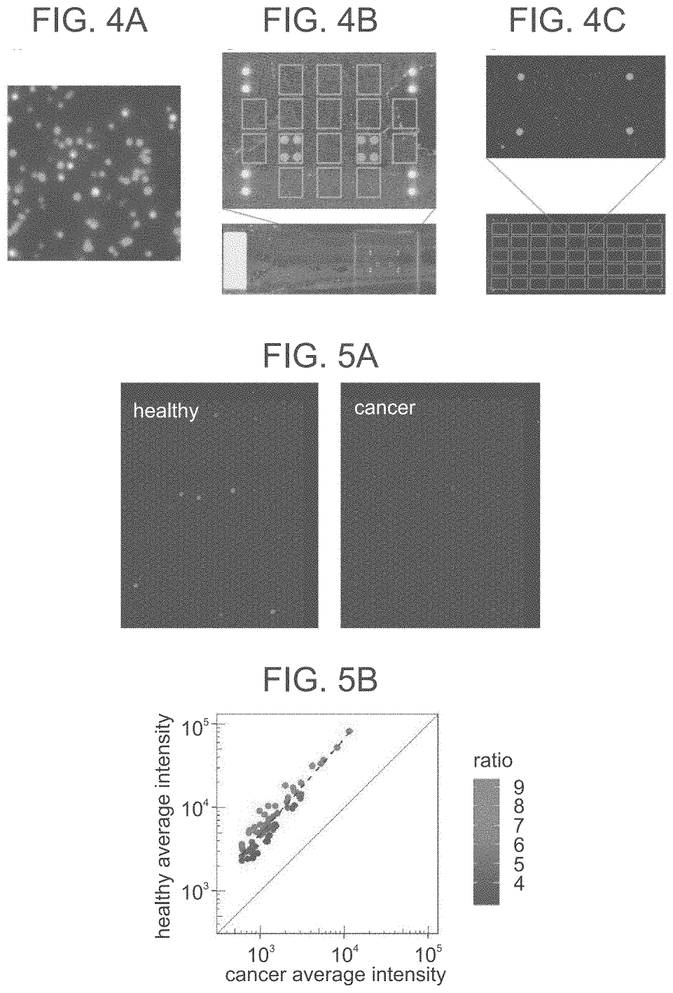

FIG. 4B shows a successful hybridization of synthetic labeled DNA signal to a customized microarray, demonstrating the feasibility of the hybridization procedure and emphasizing the high specificity of the described method. Additionally, labeled cfDNA was successfully hybridized to a customized microarray (FIG. 4C), indicating the method is sensitive and can be directly performed on cfDNA. Moreover, the data indicates the method is highly specific in terms of hybridization to the complementary probe.

### Example 1

### Cancer Diagnosis Using 5HMC-Labeled DNA Hybridized to a Microarray

### Experimental Procedure

Colon DNA samples from biopsies obtained from two cancer patients and two healthy individuals were analyzed in duplicate on eight DNA microarrays (Agilent ISCA 8×60K v2 array slide). These arrays are originally meant for comparative genomic hybridization (aCGH). Each of the arrays contained 60,000 different probe sequences. The colon DNA samples were sheared to fragments of approximately 1000-1300 bp in length. 5-hydroxymethyl cytosines (5hmC) in the resulting DNA fragments were enzymatically labeled with the red fluorophore Cy5, using glycosyltransferase and a modified cofactor, followed by copper-free click chemistry, as described in 13-14 and Michaeli et al. Clem Commun (Camb). (2013) 49(77):8599-601. Each of the four labeled DNA samples was split in half to produce duplicates and then separately hybridized to an Agilent ISCA 8×60K v2 array slide. The slide was scanned on a slide scanner and the resulting image was analyzed with Agilent's Feature Extraction software, yielding the fluorescence intensity values for each of the 60,000 probes.

### Results

Most of the array spots remained dark in all arrays, indicating low levels of 5hmC in most genomic regions. However, many of the spots on the array yielded a bright fluorescent signal when hybridized with DNA from healthy individuals. In contrast, the arrays hybridized with DNA from cancer patients yielded a fluorescent signal in fewer spots and with lower intensities. Hence, one can clearly differentiate between the arrays hybridized with DNA from healthy individuals from the arrays hybridized with DNA from cancer patients (FIG. 5A). The technical and the biological replicates showed a good correlation and thereby demonstrate a good reproducibility (also confirmed by cluster analysis and principle component analysis). FIG. 5B shows the average intensities of array spots that were particularly good in differentiating between healthy and cancer DNA.

### Example 2

### Detection of Unmethylated DNA at a Concentration of 1 fg/μl Hybridized to an Array

### Experimental Procedure

Capture array preparation—2 mm diameter holes in a custom hydrophobic adhesive tape were cut and the tape glued to a 2D-Epoxy PolyAn functionalized coverslip (PolyAn Cat No. 104 00 226) in order to form hydrophobic boundaries forming a grid divided into separated cells.

Following, 2.5 μl of 300 ng/μl of capture probes (/5AmMC12/-PTP, IDT, SEQ ID NO: 1) in NEXTERION SPOT buffer (0.25 M Na₂HPO₄ pH 9, 2.2% (w/V) Na₂SO₄) were applied to the exposed surface of the coverslip (i.e. inside each of the grid cells). The coverslip was incubated at 42° C. for 14 minutes and then at 30° C. for 20 minutes. Following incubation, the coverslip was washed in a 50 ml falcon tube with DDW by manually inverting the tube 100 times. The coverslip was transferred to another 50 ml falcon tube, containing ethylene glycol (Sigma-Aldrich) solution with a ratio of 1:4 (V/V in DDW), and incubated at 37° C. for 1 hour with shaking. Following incubation, the coverslip was transferred to a 50 ml falcon tube containing 3% FBS (Glibco) solution (V/V in DDW) and incubated at 37° C. for 2 hours with shaking. Next, the coverslip was transferred to a 50 ml falcon tube with DDW and washed by manually inverting the tube 100 times. The washing step was repeated again with a fresh 50 ml falcon tube containing DDW. Following the second wash, the coverslip was blow dried with nitrogen.

Hybridization—Probes complementary (PTP, SEQ ID NO: 2) or non-complementary (NXEP4, SEQ ID NO: 3) to the capture probes were fluorescently labeled according to 13-14 and Michaeli et al. Chem Commun (Camb). (2013) 49(77):8599-601, yielding 5Alex647N/-PTP and 5Alex647N/-NXEP4, respectively, representing a tested DNA sample. Following, solutions containing 10 fg/μl or 1 fg/μl of 5Alex647N/-PTP or 5Alex647N/-NXEP4 were prepared in hybridization buffer (3× SSC buffer, Sigma-Aldrich, 0.25% SDS, Bio-Lab tld.); and 1.5 μl of these solutions were applied to the grid cells created on the coverslips (inside the cells), each solution to a separate coverslip. The coverslips were incubated at 42° C. for 14 minutes and then at 30° C. for 20 minutes. Following incubation each coverslip was transferred into a 50 ml falcon tube containing wash solution A (0.6× SSC, 0.02% SDS) and washed by manually inverting the tube 100 times. The washing step was repeated again with a fresh 50 ml falcon tube containing wash solution A. Following, the coverslip was transferred into a 50 ml falcon tube containing wash solution B (0.03× SSC) and washed by manually inverting the tube 100 times. The washing step was repeated again with a fresh 50 ml falcon tube containing wash solution B (0.03× SSC).

Optical detection and analysis - Following the last wash, the coverslips were blow-dried with nitrogen and imaged in an epi-fluorescence microscope with a magnification of 150× in oil immersion. To quantitate, several fields of view (FOVs) were acquired for each sample and analyzed by a custom software that counts the number of spots in each image using a specified threshold.

### Results

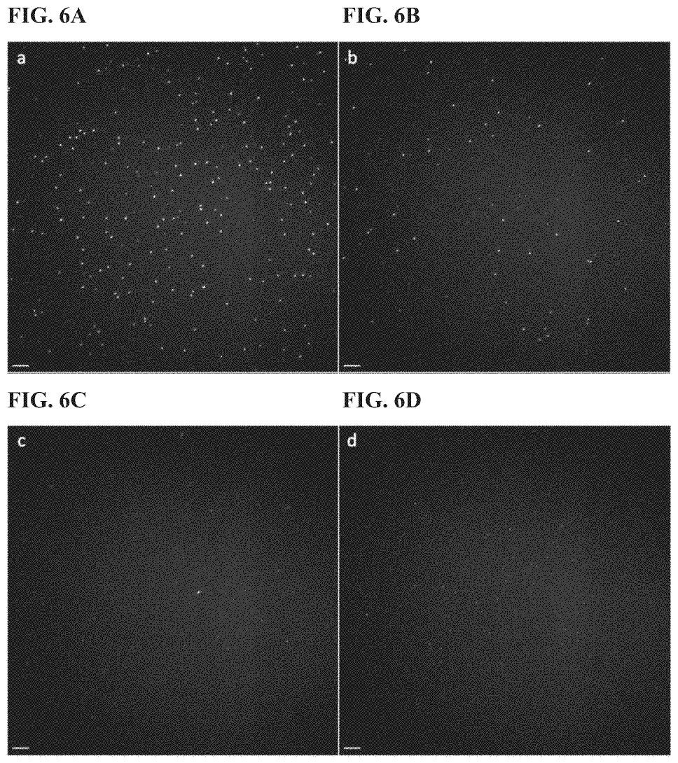

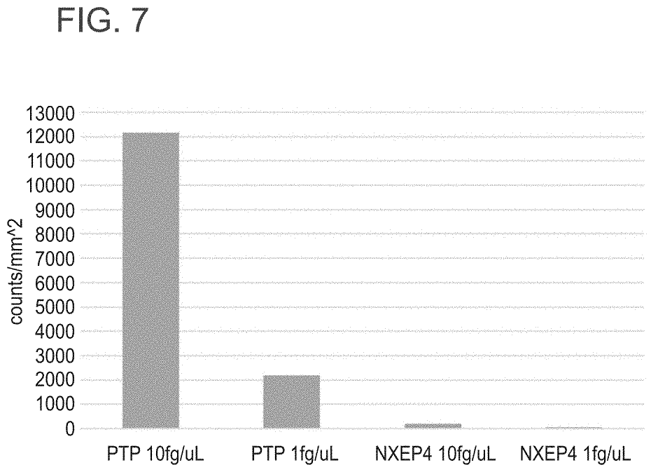

Most of the commercially available microarrays slides are not sensitive enough to analyze minute amounts of DNA. For example, the concentration of a specific target in the cell-free DNA of a sick individual can change very slightly, so that without amplification techniques, commercially available devices cannot detect it due to high non-specific background. To this end, the present inventors utilized 2D epoxy coated coverslips with 0.1 mm thickness and single-molecule fluorescence imaging to increase the limit of detection by several orders of magnitude by almost completely eliminating background noise. Specifically, a single-pixel capture surface for the cell-free target PTPrcap was created and its' capture capabilities for two cell-free targets (PTPrcap and NXEP4), known to be unmethylated and therefore fluorescently labeled in all samples, was test. As median cell-free concentration in healthy individuals is ˜0.05 ng/μl, the ability to detect down to 1 copy of target in 50 million (target concentration of 1 fg/μl) was tested. As shown in FIG. 6, detection was extremely specific to the complementary PTPrcap DNA even down to a concentration of 1 fg/μl which resulted in over 2000 capture events per mm². The non-complementary fluorescent NXEP4 DNA resulted in ˜20 capture events for the same concentration, similar to the background level, indicating no false positive detection. To get quantitative results, multiple microscope FOVs were acquired for each sample and analyzed by a custom software that counts the number of spots in each image using a specified threshold. FIG. 7 summarizes the results for the various samples, where the y-axis represents the counts per 1 mm² in each sample.

Taken together, the results indicate that target DNA at concentrations down to 1 fg/μl are easily detected, counted and discriminated from the background and from non-specific DNA sequences without the need for amplification.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. American Cancer Society. Cancer Treatment and Survivorship Facts & Figs. 2016-2017. American Cancer Society, Atlanta; 2016
2. Cancer Research UK. Breast cancer survival statistics (2015)
3. Lo, Y M Dennis, et al. “Plasma DNA as a prognostic marker in trauma patients.” Clinical chemistry 46.3 (2000): 319-323.
4. Rainer, Timothy H., et al. “Prognostic use of circulating plasma nucleic acid concentrations in patients with acute stroke.” Clinical chemistry 49.4 (2003): 562-569.
5. Lehmann-Werman, Roni, et al. “Identification of tissue-specific cell death using methylation patterns of circulating DNA.” Proceedings of the National Academy of Sciences 113.13 (2016): E1826-E1834.
6. Feng, Suhua, et al. “Conservation and divergence of methylation patterning in plants and animals.” Proceedings of the National Academy of Sciences 107.19 (2010): 8689-8694.
7. Ehrlich, Melanie, et al. “Amount and distribution of 5-methylcytosine in human DNA from different types of tissues or cells.” Nucleic acids research 10.8 (1982): 2709-2721.
8. Zeng, Hu, et al. “Liquid biopsies: DNA methylation analyses in circulating cell free DNA.” Journal of Genetics and Genomics (2018).
9. Frommer, Marianne, et al. “A genomic sequencing protocol that yields a positive display of 5-methylcytosine residues in individual DNA strands.” Proceedings of the National Academy of Sciences 89.5 (1992): 1827-1831.
10. Herman, James G., et al. “Methylation-specific PCR: a novel PCR assay for methylation status of CpG islands.” Proceedings of the national academy of sciences 93.18 (1996): 9821-9826.
11. Bibikova, Marina, et al. “High density DNA methylation array with single CpG site resolution.” Genomics 98.4 (2011): 288-295.
12. Bardelli, Alberto, and Klaus Pantel. “Liquid biopsies, what we do not know (yet).” Cancer cell 31.2 (2017): 172-179.
13. Gilboa, Tal, et al. “Single-molecule DNA methylation quantification using electro-optical sensing in solid-state nanopores.” ACS nano 10.9 (2016): 8861-8870.
14. Grunwald, Assaf, et al. “Reduced representation optical methylation mapping (R2OM2).” bioRxiv (2017): 113522.
15. Jain, Nikhil et al “Global modulation in DNA epigenetics during pro-inflammatory macrophage activation” Epigenetics (2019), 14:12, 1183-1193

1. A method of identifying DNA having an epigenetic pattern distinctive of a cell or tissue type or state, the method comprising:
(a) labeling an epigenetic modification of interest in a DNA sample with a label;
(b) contacting said sample on an array comprising a plurality of probes for said DNA under conditions which allow specific hybridization between said plurality of probes and said DNA; and
(c) detecting said hybridization, wherein an amount of said label is indicative of the cell or tissue type or state, wherein the method is effected in the absence of amplification of said DNA.

2. The method of claim 1, wherein said epigenetic modification of interest is represented by a plurality of different DNA fragments.

3-4. (canceled)

5. A method of identifying DNA having an epigenetic pattern distinctive of a cell or tissue type or state, the method comprising:
(a) labeling an epigenetic modification of interest in a sample comprising DNA with a label such that said epigenetic modification of interest is represented by a plurality of different DNA fragments;
(b) contacting said sample on an array comprising probes for said DNA fragments under conditions which allow specific hybridization between said probes and said DNA, wherein said array is designed such that a plurality of different probes for said plurality of different DNA fragments are positioned on a single grid cell of said array; and
(c) detecting said hybridization, wherein an amount of said label per said single grid cell of said array is indicative of the cell or tissue type or state.

6. The method of claim 5, wherein the method is effected in the absence of amplification of said DNA and said DNA fragments.

7-10. (canceled)

11. The method of claim 1, wherein a concentration of said DNA in said sample is ≤10 ng/μl.

12. (canceled)

13. The method of claim 1, wherein a concentration of said DNA in said sample is ≤10 fg/μl.

14. (canceled)

15. The method of claim 1, wherein said array comprises a glass having a thickness ≤250 μm.

16. The method of claim 1, wherein said array comprises a glass featuring a functionalized group capable of binding said -probe.

17-19. (canceled)

20. The method of claim 16, wherein said functional group is capable of covalently binding said probe.

21. The method of claim 20, wherein said functional group is an epoxide.

22. The method of claim 1, wherein said array allows the use of an oil immersion microscope objective for imaging of said array.

23-24. (canceled)

25. The method of claim 1, wherein the method is effected in the absence of bisulfite conversion and/or sequencing.

26-27. (canceled)

28. The method of claim 1, wherein said DNA is cell-free DNA (cfDNA).

29-32. (canceled)

33. A method of diagnosing a pathology in a subject, the method comprising obtaining a biological sample of the subject and identifying DNA having an epigenetic pattern distinctive of a cell or tissue type or state according to the method of claim 1, wherein presence and/or level above a predetermined threshold of said DNA having said epigenetic pattern distinctive of said cell or tissue type or state is indicative of a pathology associated with said cell or tissue in said subject.

34. A method of treating a pathology in a subject in need thereof, the method comprising:
(i) diagnosing the pathology in the subject according to the method of claim 33; and wherein said pathology is indicated
(ii) treating said pathology in said subject.

35. A method of monitoring a treatment for a pathology in a subject in need thereof, the method comprising obtaining a biological sample of the subject and identifying DNA having an epigenetic pattern distinctive of a cell or tissue associated with the pathology according to the method of claim 1, wherein a decrease above a predetermined threshold of said DNA having said epigenetic pattern distinctive of said cell or tissue following treatment as compared to same prior to treatment indicates efficacy of treatment of the pathology in said subject.

36. (canceled)

37. A method of detecting death of a cell or tissue of interest in a subject comprising determining whether cell-free DNA (cfDNA) comprised in a fluid sample of the subject is derived from the cell or tissue of interest, wherein said determining is effected by the method of claim 1, wherein presence and/or level above a predetermined threshold of said DNA having an epigenetic pattern distinctive of said cell or tissue of interest is indicative of death of the cell or tissue of interest.

38-44. (canceled)

45. An array comprising a plurality of different probes for a plurality of different nucleic acid sequences positioned on a single grid cell of the array.

46-49. (canceled)

50. A kit comprising the array of claim 45; and a label, a positive control template comprising said nucleic acid sequences and/or an enzyme for labeling said nucleic acid sequences.

51-55. (canceled)

56. The method of claim 1, wherein said epigenetic modification comprises unmethylated CpG; or wherein said epigenetic modification comprises 5-methylcytosine (5mC) and/or 5-hydroxymethylcytosine (5hmC).

57-61. (canceled)


SEQUENCE LISTING (3 sequences)

SEQ ID NO: 1 - DNA, 60 bp, Artificial Sequence
  complementary PTP-Alexa647N
  misc_feature (1)..(60): conjugated to Alexa647N
ggggggctccgaggcttaccattggtccgcaggcccctccgtgccgggcacccacctcca

SEQ ID NO: 2 - DNA, 60 bp, Artificial Sequence
  PTP capture probe
tggaggtgggtgcccggcacggaggggcctgcggaccaatggtaagcctcggagcccccc

SEQ ID NO: 3 - DNA, 60 bp, Artificial Sequence
  non-complementary NXEP4-Alexa647N
  misc_feature (1)..(60): conjugated to Alexa647N
ggaaagattgatgggcatgcttaaataagatggaaagatgatgggcatgccctcctgtgt